(19) United States
(12) Patent Application Publication
(10) Pub. No.: US 2007/0173838 A1
(43) Pub. Date: Jul. 26, 2007

(54) Femoral head and neck strengthening device

(76) Inventor: Kung-Chia Li, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/330,126

(22) Filed: Jan. 12, 2006

(51) Int. Cl. A61B 17/56

(52) U.S. Cl. 606/67

(57) ABSTRACT

A femoral head and neck strengthening device includes a first strengthening element, a second strengthening element joined to the first strengthening element in a separable manner, and a bone plate joined to the second strengthening element in a separable manner; the first strengthening element can be used alone; the first strengthening element also can be used after it is joined to the second strengthening element, and a central connecting rod is passed through both the strengthening elements; the strengthening device also can be used with the first and the second strengthening elements and the bone plate being joined together, and with the bone plate being fixed to an outer side of a femur, thus increasing the supporting strength of the head of the femur as well as fixing peritrochanteric fracture or even proximal femur fracture.

### BACKGROUND OF THE INVENTION

1. Field of the Invention

The present invention relates to a femoral head and neck strengthening device, more particularly one, which includes several separable sections each used for strengthening one portion of a fractured femur, and which can be used for strengthening support of a femoral head, fixing femoral neck fracture trochanteric fracture.

2. Brief Description of the Prior Art

The known causes of a vascular necrosis of femoral head are roughly grouped into injury-related and non-injury-related ones. The injury-related causes of a vascular necrosis of femoral head can be related with the patient's femoral neck fracture or dislocation of hip joint, which will result in injury of blood vessels that supply the femoral head, and a vascular necrosis of the femoral head. The non-injury-related causes of a vascular necrosis of femoral head can be drinking alcohol, smoking, overheavy workload or corticosteroid.

The evolution process of a vascular necrosis of femoral head includes six stages:

1. stage one: the patient feel pain. Radiographs reveal normal bony structure. Necrosis can only be seen through MRI. At this stage, the condition is handled through medication;

2. stage two: cystic change of a femoral head is visible, and the condition is handled through core decompression;

3. stage three: subchondral bone of a femoral head caves in, but the femoral head hasn't flattened yet;

4. stage four: the femoral head caves in and flattens obviously, and hip hemi-arthroplasty is used;

5. stage five: the hip joint degenerates and narrows;

6. stage six: the acetabulum structure of the pelvis is damaged, and the whole hip joint degenerates to a serious condition; at stages five and six, the condition has to be handed with a total hip replacement.

At stages two and three of a vascular necrosis of femoral head, if core decompression alone can't prevent the femoral head from collapse, other artificial devices should be planted in addition. Therefore, there is need for suitable femoral head strengthening devices.

Furthermore, the elderly are prone to suffer from a vascular necrosis of femoral head and fracture of a near-end portion of the femur owing to bone fragility. Referring to FIG. 9, a currently existing dynamic hip screw (DHS) is used to fix trochanteric fracture 91. The dynamic hip screw includes a bone plate 92, a bolt 93, and several bone screws 94; the bolt 93 is inserted into the head 911 of the femur 91 through the bone plate 92 and an outer side of the femur 91; the bone screws 94 fix the bone plate 92 to an outward side of the femur 91. The above structure has the following advantages: the wound resulting from surgery is relatively small, and the length of time used in surgery is relatively short. However, some of the parts of the dynamic hip screw won't be needed in using the dynamic hip screw to increase the supporting strength of the femoral head 911, and there will be waste of materials and time in surgery if the patient only suffers a vascular necrosis of femoral head.

The inventor of the present invention has developed a femoral head and neck strengthening device, which includes several separable sections each used for strengthening one portion of a fractured femur such that it can be used for handling a vascular necrosis of a femoral head, and also can be used for fixing a femoral neck or trochanteric fracture.

### SUMMARY OF THE INVENTION

It is a main object of the invention to provide a femoral head and neck strengthening device for strengthening support for a femoral head.

It is a secondary object of the invention to provide a femoral head and neck strengthening device for fixing the femoral neck fracture or trochanteric fracture.

The femoral head and neck strengthening device of the present invention includes a first strengthening element, a second strengthening element, a bone plate, first and second dividable coupling mechanisms, and a central connecting rod; the first dividable coupling mechanism is connected to both first ends of the first and the second strengthening elements such that the strengthening elements can be coupled in a separable manner; the second dividable coupling mechanism is connected to both a second end of the second strengthening element and the bone plate such that the second strengthening element and the bone plate can be coupled in a separable manner; the central connecting rod is passed through both the second strengthening element and the bone plate and fixed in a connecting hole of the first strengthening element.

The strengthening device can be used in a first manner, in which the first strengthening element is planted into a femoral head alone for strengthening the support of the femoral head. The strengthening device can be used in a second manner, in which the first and the second strengthening elements are coupled and planted into a fractured neck of a femur for fixation. The strengthening device can be used in a third manner, in which the first and the second strengthening elements and the bone plate are joined together, and the bone plate is fixed to a femur by means of at least one fixing element passed through one of several holes on the bone plate, providing fixation to a femoral neck fracture, and peritrochanteric fracture.

### BRIEF DESCRIPTION OF THE DRAWINGS

The present invention will be better understood by referring to the accompanying drawings, wherein:

FIG. 1 is an exploded perspective view of the first preferred embodiment of the invention,

FIG. 2 is a side view of the first preferred embodiment,

FIG. 3 is an exploded perspective view of a strengthening device with the first and the second strengthening elements in the invention,

FIG. 4 is a side view of a strengthening device with the first and the second strengthening elements in the invention,

FIG. 5 is a side view showing the first manner to use the invention,

FIG. 6 is a side view showing the second manner to use the invention,

FIG. 7 is a side view showing the third manner to use the invention,

FIG. 8 is a view of the second preferred embodiment of the present invention, and

FIG. 9 is a view of the currently existing dynamic hip screw.

### DETAILED DESCRIPTION OF THE PREFERRED EMBODIMENTS

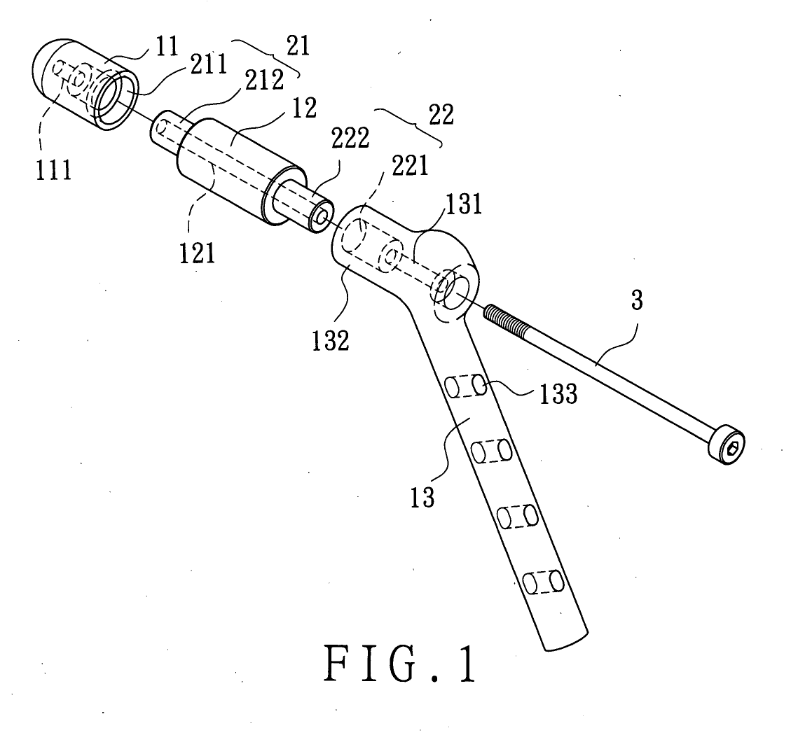

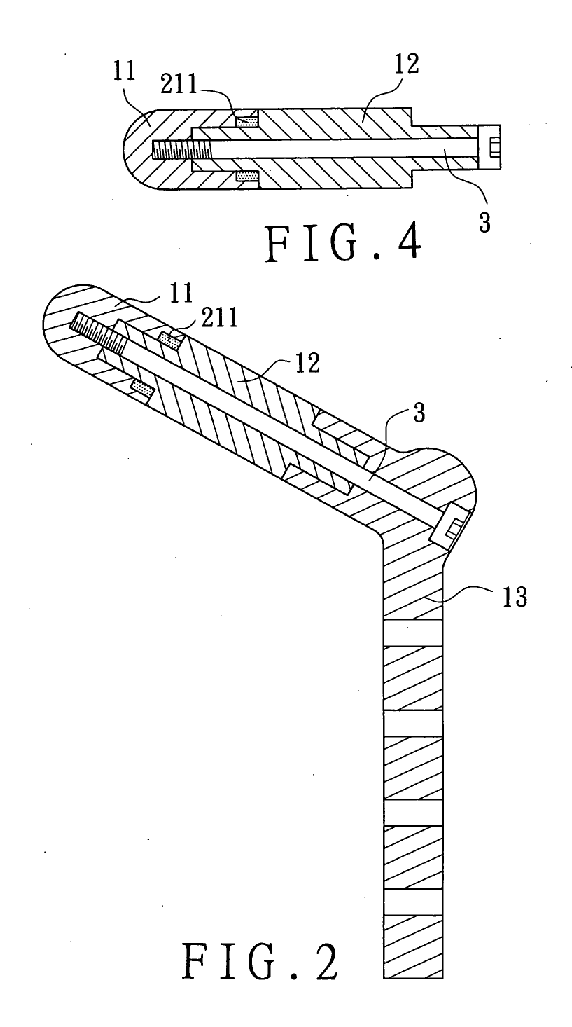

Referring to FIGS. 1 and 2, a first preferred embodiment of a femoral head and neck strengthening device in the present invention includes a first strengthening element 11, a second strengthening element 12, a bone plate 13, a first dividable coupling mechanism 21, a second dividable coupling mechanism 22, and a central connecting rod 3.

The first strengthening element 11 has a connecting hole 111, and a coarse surface for allowing bone mass to adhere to and grow on while the second strengthening element 12 has a through hole 121. Further, the first strengthening element 11 has a first end, and a second end, which has an outward curve. The bone plate 13 has a bone plate portion, a fixing seat portion 132 projecting from one end of the bone plate portion, a hole 131 through the fixing seat portion 132, and several through holes 133 on the bone plate portion; the angle between the bone plate portion and the fixing seat portion 132 of the bone plate 13 is normally one hundred and thirty-five to one hundred and fifty degrees in order to suit the angle of a human femoral neck.

The first dividable coupling mechanism 21 includes a hollow part 211, and a protruding part 212. The hollow part 211 and the protruding part 212 are held in the first end of the first strengthening element 11, and joined to one end of the second strengthening element 12 respectively, and the protruding part 212 can be fitted in the hollow part 211 in a separable manner; thus, the first strengthening element 11 can be joined to the second strengthening element 12 in a separable manner by means of the first coupling mechanism 21.

The second dividable coupling mechanism 22 includes a hollow part 221, and a protruding part 222. The hollow part 221 and the protruding part 222 are held in one end of the fixing seat portion 132 of the bone plate 13, and joined to the other end of the second strengthening element 12 respectively, and the protruding part 222 can be fitted in the hollow part 221 in a separable manner; thus, the second strengthening element 12 can be joined to the bone plate 13 in a separable manner by means of the second coupling mechanism 22.

The first and the second strengthening elements 11, 12, and the bone plate 13 are joined together, and the central connecting rod 3 is passed through the through holes 121 and 131, and fixed in the connecting hole 111 to securely connect the first and the second strengthening elements 11, 12, and the bone plate 13.

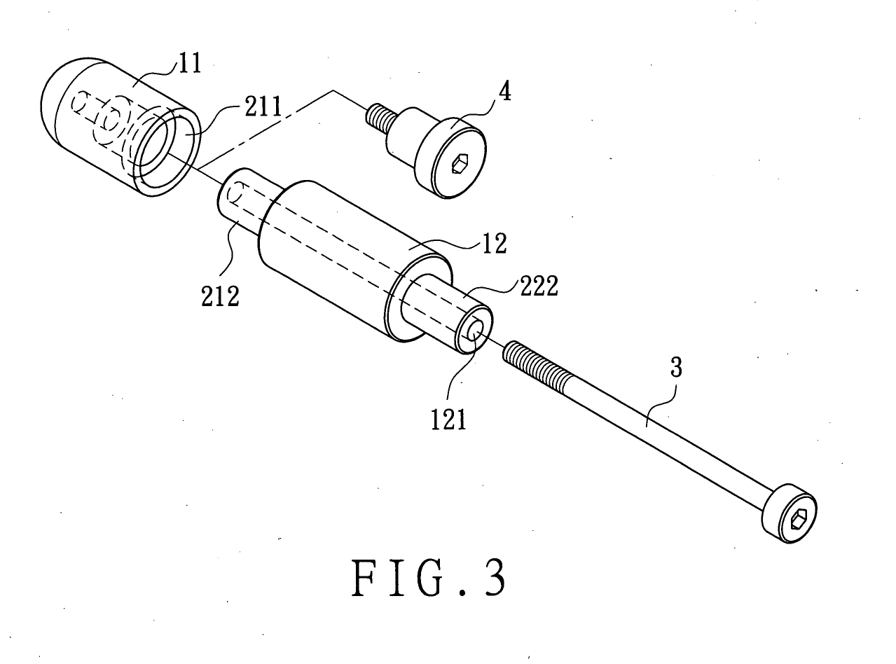

The first strengthening element 11 of the strengthening device can be put into clinic use alone: in order to fill the hollow part 211 in the first strengthening element 11, the present invention is further equipped with a post-shaped plug 4. The first and the second strengthening elements 11 and 12 together can be put into clinical use for fixation of a near-end fractured portion of a femur after they are fastened together by means of a suitable-lengthed central connecting rod 3, as shown in FIGS. 3 and 4.

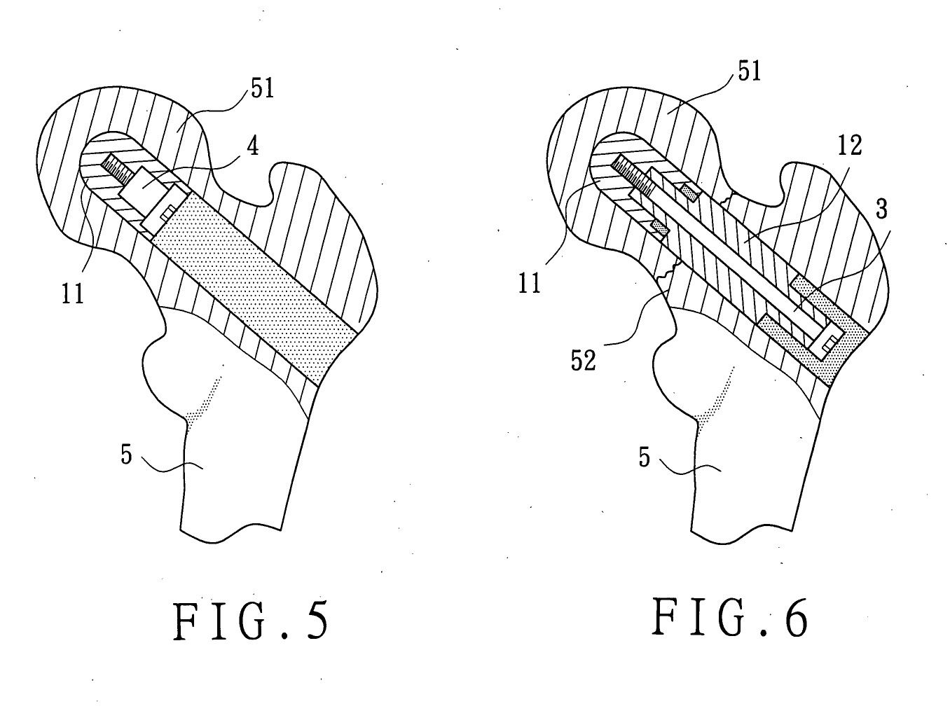

FIGS. 1 and 5 show the strengthening device of the present invention put into clinical use in a first manner: if a femoral head 51 of a femur 5 loses its supporting strength owing to a vascular necrosis, it is prone to collapse when subjected to force. To handle this problem, the first strengthening element 11 is planted into the femoral head 51 alone, and the post-shaped plug 4 is threadedly inserted into the hollow part 211 inside the first strengthening element 11, thus increasing the supporting strength of the femoral head 51 as well as lengthening the service life of the femoral head 51.

FIGS. 1 and 6 show the strengthening device of the present invention put into clinical use in a second manner: if a femoral neck 52 of a femur 5 breaks, and a femoral head 51 of the femur 5 loses its supporting strength owing to a vascular necrosis, the first and the second strengthening elements 11 and 12 joined together are passed through the femoral neck 52 and inserted into the femoral head 51 after the strengthening elements 11 and 12 are secured together by means of the central connecting rod 3 with the protruding part 212 being fitted into the hollow part 211 of the first coupling mechanism 21; thus, the first strengthening element 11 is secured in the femoral head 51 for allowing the femoral neck 52 to heal. The patient's body and muscle will exert gravitational force and pulling force on the femoral head 51, which will be passed on to the present strengthening device and the femur 5 via the surface of the femoral head 51, thus helping the two opposed sides of the broken portion of the femoral neck 52, get in touch with each other. Consequently, growth of bone mass and post-surgery recovery will be more rapid.

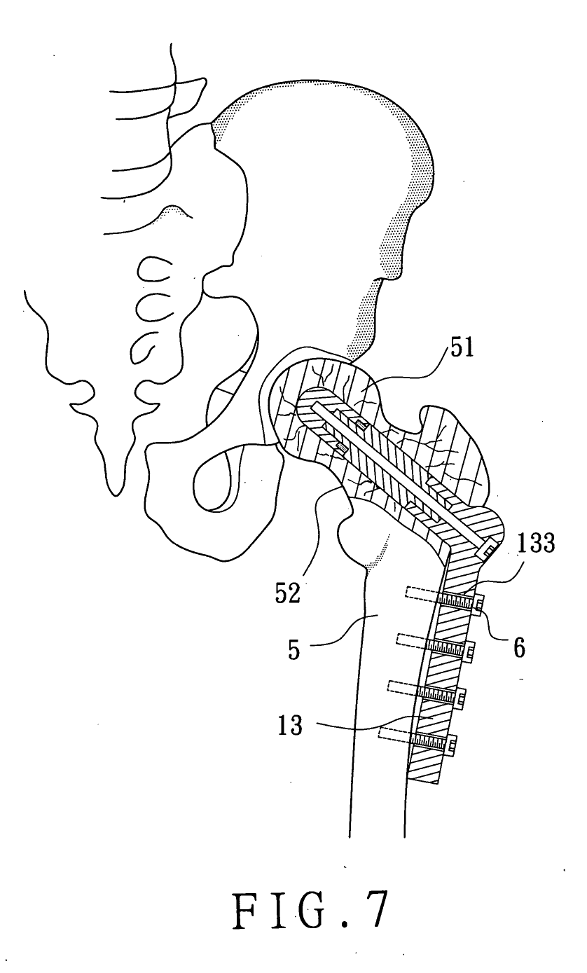

FIGS. 1 and 7 show the strengthening device of the present invention put into clinical use in a third manner: if a femoral head 51 or a femoral neck 52 of a femur 5 breaks to such an extent as to lose its completeness, and needs further strengthening, the first and the second strengthening elements 11, 12 and the bone plate 13 joined together are fixed to the femur 5 with the strengthening elements 11 and 12 being passed through the femoral neck 52 and inserted into the femoral head 51, and with the bone plate 13 being secured to the femoral shank of the femur 5 by means of at least one fixing element 6, which is passed through one of the through holes 133 of the bone plate 13 and fixed in the femoral shank; thus, the incomplete femoral head 51 (femoral neck 52) is strengthened and supported. In the present preferred embodiment, the fixing element 6 is a bone screw.

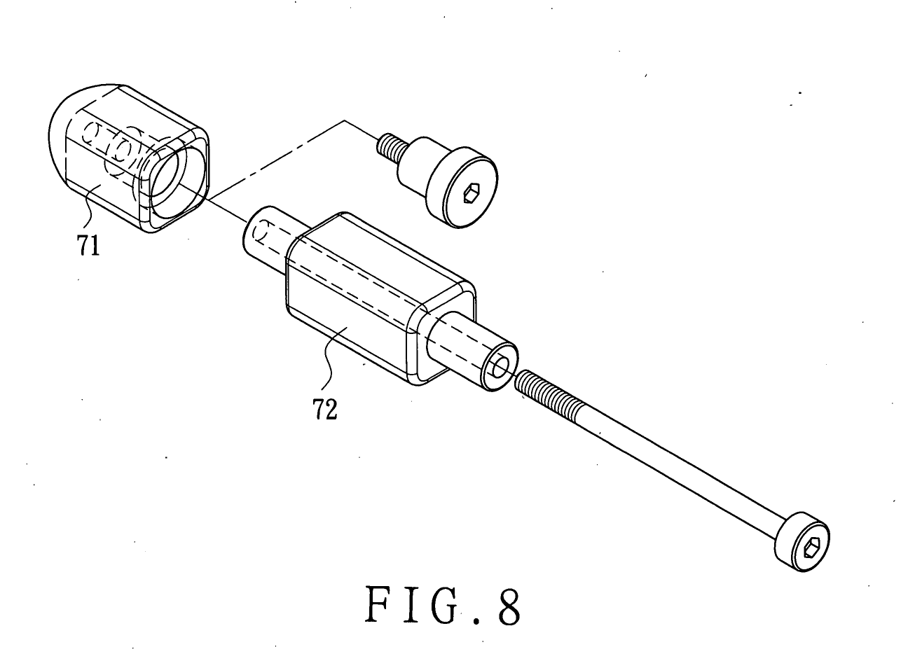

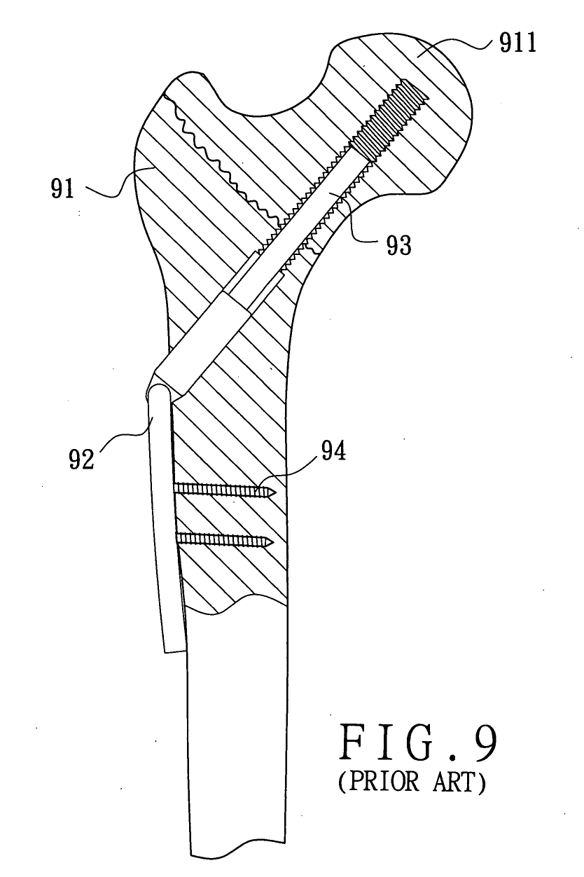

The first and the second strengthening elements 11 and 12 can be in the shape of a cylinder as shown in FIG. 3, and they can be formed in such a shape that their cross section is rectangular or square, as shown in FIG. 8.

From the above description, it can be easily seen that the present invention has the following advantages:

1. The strengthening devices of the present invention can be used to handle many different problems such as a vascular necrosis of femoral head, breaking of femoral neck, and trochanteric fracture. An appropriate strengthening device can be chosen and used according to the type of the operation.

2. The present invention will provide rotational and axial stability, thus providing a fractured bone with desirable dynamic axial pressure. Therefore, the length of time the patient in bed will reduce, and the patient will recover and become capable of bearing weight again sooner.

3. The strengthening devices of the present invention have simple structure, and they are easy to manufacture and suitable for use in many different situations.

What is claimed is:

1. A femoral head and neck strengthening device, comprising
a first strengthening element having a connecting hole;
a second strengthening element having a through hole;
a bone plate having a through hole;
a first dividable coupling mechanism connected to both first ends of the first and the second strengthening elements such that the first and the second strengthening elements can be coupled in a separable manner;
a second dividable coupling mechanism connected to both a second end of the second strengthening element and the bone plate such that the second strengthening element and the bone plate can be coupled in a separable manner; and
a central connecting rod passed through the through holes of the second strengthening element and the bone plate and fixed in the connecting hole of the first strengthening element.

2. The femoral head and neck strengthening device as claimed in claim 1, wherein the bone plate has at least one hole for allowing a fixing element to pass through, which is fixed in a femur; the fixing element being a bone screw.

3. The femoral head and neck strengthening device as claimed in claim 1, wherein the bone plate has a fixing seat portion protruding from it, and it is joined to the second strengthening element at the fixing seat portion.

4. The femoral head and neck strengthening device as claimed in claim 3, wherein an angle between the bone plate and the fixing seat portion protruding from the bone plate suits an angle of a human femoral neck.

5. The femoral head and neck strengthening device as claimed in claim 1, wherein the first coupling mechanism includes a hollow part held in the first strengthening element, and a protruding part joined to the second strengthening element; the protruding part fitting in the hollow part in a separable manner.

6. The femoral head and neck strengthening device as claimed in claim 1, wherein the second coupling mechanism includes a hollow part held in the bone plate, and a protruding part joined to the second strengthening element and fitting in the hollow part in a separable manner.

7. The femoral head and neck strengthening device as claimed in claim 1 being further equipped with a plug, which is inserted into a hollow in the first strengthening element to fill the hollow.

8. The femoral head and neck strengthening device as claimed in claim 1, wherein the first strengthening element has a coarse surface for allowing bone mass to adhere to and grow on.

9. The femoral head and neck strengthening device as claimed in claim 1, wherein the first and the second strengthening elements joined together have a cylindrical shape.

10. The femoral head and neck strengthening device as claimed in claim 1, wherein the first and the second strengthening elements joined together have such a shape that their cross section is rectangular.
